(19) United States
(12) Patent Application Publication
(10) Pub. No.: US 2016/0000691 A1
(43) Pub. Date: Jan. 7, 2016

(54) Topical Compositions And Methods For Whitening Skin

(71) Applicant: ELC Management LLC, Melville, NY (US)

(72) Inventors: Fatemeh Mohammadi, Hauppauge, NY (US); Daniela Bratescu, Northport, NY (US)

(21) Appl. No.: 14/853,009

(22) Filed: Sep. 14, 2015

Related U.S. Application Data
(62) Division of application No. 12/863,905, filed on Aug. 17, 2010, now abandoned.
(60) Provisional application No. 61/029,967, filed on Feb. 20, 2008.

(51) Int. Cl. A61K 8/86; A61K 8/02; A61Q 19/02; A61K 8/34

(52) U.S. Cl. CPC A61K 8/86; A61K 8/347; A61K 8/0295; A61Q 19/02; A61K 2800/591

(57) ABSTRACT

Methods and compositions for whitening or brightening skin comprising at least one whitening active contained in association structures.

### TECHNICAL FIELD

The invention is in the field of topical cosmetic or pharmaceutical compositions for application to keratinous surfaces and methods for whitening or brightening skin using the compositions.

### BACKGROUND OF THE INVENTION

Skin whitening is a very popular treatment in Asian populations. In these cultures, white skin is a sign of beauty and affluence. Throughout the years many different whitening treatments have been used by Asian women who covet porcelain white skin. In the early 1900's Japanese geishas applied make up containing high concentrations of lead to whiten their skin. After years, users of these products exhibited yellow, slack, prematurely aged skin. Other ingredients such as hydroquinone, arbutin, or certain botanicals such as extracts from mulberry or bearberry are also known to whiten skin. Many of these ingredients whiten skin by inhibiting the enzyme tyrosinase, which causes the product of melanin.

In years past, skin whitening was considered predominantly of interest to Asians. However, cosmetics consumers in western countries now recognize that skin whitening (or as western consumers call it, skin brightening) is very desirable for treating skin issues such as uneven pigmentation that can arise from excessive sun exposure, age spots, freckles, and so on. Also, recent studies have shown that one hallmark of youthful skin is its luminescence. Contributing to the luminescent skin of youth is even pigmentation, healthy oxygenated skin, and an unblemished skin surface.

When it comes to ingredients that have skin whitening or brightening properties, there are two primary concerns—efficacy and skin compatibility. In particular, the whitening or brightening ingredient must work for its intended purpose and must also not be irritating to skin. In some cases, ingredients that have excellent efficacy in whitening or brightening skin can be irritating to overly sensitive skin. Also, while some ingredients exhibit skin whitening capability, they have less than optimal efficacy. Depending on the formula in which the whitening ingredient is present, it may exhibit more compatibility with the formula constituents than skin and be less than optimally absorbed into the skin. This results is less effectiveness in providing skin whitening.

It has been discovered that delivery skin whitening ingredients to the skin in the form of association structures improves efficacy and reduces any tendency for certain of such actives to be irritating to overly sensitive skin.

It is an object of the invention to provide compositions containing at least one skin whitening ingredient contained in association structures.

It is a further objective of the invention to provide a composition for whitening or brightening skin comprising at least one skin whitening agent contained in association structures.

It is a further object of the invention to provide a method for whitening or brightening skin comprising treating the skin with a composition comprising at least one skin whitening agent contained in association structures.

It is a further object of the invention to provide a method for treating uneven pigmentation, age spots, mottled or yellowed skin, skin laxity or wrinkles comprising treating the skin with a composition containing at least one skin whitening agent contained in association structures.

### SUMMARY OF THE INVENTION

The invention is directed to a composition comprising at least one skin whitening ingredient contained in association structures.

The invention is further directed to a composition for whitening or brightening skin comprising at least one skin whitening agent contained in association structures.

The invention is also directed to a method for whitening or brightening skin comprising treating the skin with a composition comprising at least one skin whitening agent contained in association structures.

The invention is also directed to a method for treating uneven pigmentation, age spots, mottled or yellowed skin, skin laxity or wrinkles comprising treating the skin with a composition containing at least one skin whitening agent contained in association structures.

The invention is also directed to a method for improving the efficacy of skin whitening ingredients by delivering such ingredients to the skin in the form of association structures contained in topical compositions.

### DETAILED DESCRIPTION

### I. Definitions

“Association structures” means a state that occurs when molecules present in a composition exhibit intermediate, as opposed to random, stages of order. For example, association structures such as vesicles or liquid crystals may be formed when certain amphiphilic ingredients present in a polar solvent-containing composition align in ordered configuration such as a tail-to-tail or head-to-head/tail-to-tail configuration. By “tail-to-tail” is meant that the hydrophilic tail portions of the molecule orient together and the lipophilic heads of the molecule orient toward the lipophilic phase of the composition. By “head-to-head/tail-to-tail” is meant that the hydrophilic portions of the amphiphilic ingredients are attracted to each other and the lipophilic portions are attracted to each other causing the amphiphilic ingredients to form a certain molecular order within the composition, which is somewhere between the completely disordered liquid state and the completely ordered solid state. Types of association structures include liposomes, liquid crystals, or vesicles such as unilamellar vesicles, large vesicles, or multilamellar vesicles, micelles, reverse micelles, and so on.

“Bright” or “Brightening” means, with respect to skin, that the skin exhibits a glow or luminescence.

“Isotropic” means a typical liquid state where molecules exhibit random order.

“Large unilamellar vesicle” means a vesicle having a single lipid layer that self-closes around the contents of the vesicle and has a diameter ranging from about 51 to 1000 nanometers.

“Liquid Crystals” means a state of molecular order in a liquid that is between the isotropic molecular order seen in a typical liquid and the structured order of molecules seen in a typical solid. In liquid crystals, amphiphilic ingredients, most often lipids, will order in head-to-head and tail-to-tail configuration such that the liquid exhibits a certain degree of molecular order despite its liquid character. Active ingredients may be incorporated into the interstices of the liquid crystal—that is, between oriented molecules.

“Liposome” means a vesicle formed from thin phospholipid films which are hydrated and the amphiphilic phospholipids orient in a tail-to-tail configuration and the lipophilic heads orient toward the outer surface or lipophilic ingredients present to form hydrated layers, wherein the phospholipid film self-closes to form a blister or phospholipid based vesicle with one external layer alone or with one external layer and one or more internal layers.

“Lyotropic” means, with respect to liquid crystals, that they are formed in a composition by the addition of a solvent.

“Micelle” means an aggregate of amphiphilic molecules in water, with the nonpolar portions in the interior and the polar portions at the exterior surface, exposed to water. Micelles often occur in water in oil emulsions where the hydrophilic portion of the amphiphilic molecules orient toward the dispersed water droplets and the nonpolar lipophilic portions of the molecules orient toward the continuous oil phase of the emulsion.

“Multilamellar vesicle” means a vesicle having multiple hydrated layers and which is self-closed, and having a diameter generally ranging from about 100 to 1000 nanometers.

“Nematic” with respect to liquid crystal, means that the liquid crystals present have no positional order but have long range orientational order, that is, that they are in a generally parallel configuration in one dimension. Nematic liquid crystals are referred to by the designation “N”. Nematic liquid crystals may be lyotropic.

“Skin brightening” means that the skin exhibits a luminescence that is achieved by inhibiting melanin production by either inhibiting the tyrosinase enzyme or inhibiting other pathways that contribute to skin melanization.

“Skin whitening” means that the skin is perceptibly whitened by inhibition of melanin production, either by inhibiting the tyrosinase enzyme or by inhibiting other pathways that contribute to melanization of skin.

“Small unilamellar vesicle” means refers to a vesicle that has a single lipid layer that self-closes and a diameter generally ranging from about 20 to 50 nanometers.

“Thermotropic” means, with respect to liquid crystals, those for which formation is dependent on temperature.

“Smectic” means, with respect to liquid crystals, that they are positionally ordered in two dimensions and may form well defined layers that in a liquid will slide over each other much like soap. Smectic liquid crystals are often given the designation “S” and may be further classified into subcategories A-H based upon their degree of ordering.

“Vesicle” means a cavity or sac that is formed from a lipid film which has been hydrated, which causes the polar lipids that are present to orient in a tail-to-tail configuration to form one or more hydrated layers, and where the lipophilic head portions of the molecule orient to the outer surface and lipophilic internal ingredients, and wherein the lipid film then self-closes to form a blister or lipid based vesicle with one layer (e.g. unilamellar), or a plurality of layers (e.g. multilamellar).

“Whitening” means, with respect to skin, that the color of the skin is perceptibly whitened by inhibition of melanin, either by blocking the enzyme tyrosinase or blocking other reactive pathways that cause melanin production.

### II. Association Structures

The composition of the invention comprises one or more whitening active ingredients contained in association structures. Such association structures include, but are not limited to those set forth herein. The composition of the invention may comprise from about 0.001 to 95%, preferably from about 0.005 to 90%, more preferably from about 0.01 to 85% by weigh of the total composition of association structures containing the active whitening ingredient.

A. Vesicles

Suitable association structures that may be used to contain the active whitening ingredient are vesicles. Such vesicles may be phospholipid based, in which case they are often referred to as liposomes. The vesicles may also be made from lipids or modified lipids that are not phospholipid based. The lipids selected must have amphiphilic properties such that a portion of the lipid has hydrophilic character and the other portion of the lipid has lipophilic character. A wide variety of lipids are suitable so long as they have amphiphilic properties and will orient in at least tail-to-tail configuration when hydrated, e.g. the hydrophilic tails of the amphiphilic ingredient will orient together and the lipophilic heads of the amphiphilic ingredient will orient together to form the outer surface of the vesicle or orient with the lipophilic ingredients present internally. Examples lipids that may be used to make vesicles include lecithin or various types of unsaturated or saturated phospholipids including those that have been enzymatically modified (e.g. lysophospholipids). Generally there are two types of phospholipids: phosphoglycerides and sphingomyelins. Phosphoglycerides are molecules where the carboxyl group of each fatty acid is esterified to the hydroxyl groups on carbon 1 and 2 of the glycerol molecule, and where the phosphate group is attached to the third carbon atom by an ester link. Examples of phosphoglycerides that may be used to prepare vesicles include hydrogenated or nonhydrogenated phosphatides such as phosphatidylcholine, phosphatidylserine, phosphatidylethanolamine, phosphatidyl inositol, diphosphatidyl glycerol and so on. Sphingomyelins may also be used to prepare vesicles. Sphingomyelins have a sphingosine backbone. Ingredients suitable for the preparation of liposomes or vesicles may be purchased from Lipoid GmbH, Frigenstrasse 4, D-67065, Ludwigshafen, Germany as well as other cosmetic vendors that sell similar types of ingredients. Liposomes may be prepared by preparing aqueous dispersions of large multilamellar vesicles by dissolving the lipid in organic solvent, adding water to hydrate, and detaching the lipid sheets formed so they self-close to form large multilamellar vesicles.

Also, nonphospholipid amphiphilic ingredients are suitable for forming vesicles. Such ingredients are typically amphiphilic lipids that hydrate to form layers upon introduction of water or polar solvents such as alcohol, then self close to form a blister or sac. Such amphiphilic lipids may include alkoxylated fatty carboxylic acid mono-, di-, or triesters; alkoxylated glycerolated fatty mono-, di-, or triesters, sulfonated fatty acid mono-, di-, or triesters, and so on. Examples of alkoxylated fatty esters include those having from about 2 to 500 alkoxy, preferably ethoxy groups, which confer hydrophilicity. Examples include PEG (polyethylene glycol) having repeating ethylene oxide units ranging from 2 to 500. The fatty acid esters may be mono-, di-, or triesters, and if di-, or triesters, reacted with alkoxylated and glycerolated moieties. In one preferred embodiment the alkoxylated fatty acid esters or alkoxylated glycerolated fatty acid esters wherein the fatty acid is an aliphatic carbon chain ranging from about 4 to 30 carbon atoms. Examples of such fatty acid esters include, but are not limited to, monoesters of PEG and fatty carboxylic acids, diesters of PEG and fatty carboxylic acids, or triesters of PEG and fatty carboxylic acids; diesters of PEG, glycerin, and fatty carboxylic acids; triesters of PEG, glycerin, and fatty carboxylic acids. Examples of such molecules include PEG butyrate, PEG isobutyrate, PEG pentanoate, PEG hexanoate, PEG dihexanoate, PEG heptanoate, PEG diheptanoate, PEG octanoate, PEG dioctanoate, PEG nonanoate, PEG dinonanoate, PEG decanoate, PEG dodecanoate, PEG stearate, PEG distearate, PEG isostearate, PEG diisostearate, PEG laurate, PEG dilaurate, PEG myristate, PEG dimyristate, PEG behenate, PEG oleate, PEG dioleate, PEG linoleate, PEG dilinoleate, and so on. Also suitable are esters of glycerin, PEG, and fatty carboxylic acids, such as PEG glycerol dibutyrate, PEG glycerol dipentanoate, PEG glycerol dihexanoate, PEG glyceryl diheptanoate, PEG glycerol dioctanoate, PEG glycerol dinonanoate, PEG glyceryl didecanoate, PEG glyceryl distearate, PEG glyceryl diisostearate, PEG glycerol dilaurate, PEG glycerol dimyristate, PEG glyceryl dibehenate, PEG glyceryl dioleate, PEG glycerol dilinoleate, and so one. In the examples mentioned above, the number of repeating ethylene oxide moieties may range from 1 to 500 (e.g PEG₁₋₅₀₀) and, if desired, the number of glycerol moieties may range from 1 to 500, but the molecule should contain enough ethylene oxide and/or glycerol moieties to confer the necessary hydrophilic character to at least a portion of the molecule.

Also suitable for formation of vesicles are fatty alkoxylated alcohols include those having from about 4 to 30 carbon atoms in the fatty chain, which may be saturated or unsaturated. Examples of preferred alkoxylated alcohols include steareth, ceteth, ceteareth, beheneth, and the like, having from 1 to 200 repeating ethylene oxide moieties.

Sorbitan derivatives are also suitable for forming non-phospholipid vesicles. Suitable sorbitan derivatives include esters or ethers of sorbitan, which is a heterocyclic ether formed by the dehydration of sorbitol. Sorbitan may be derivatized by ethoxylation and/or esterification of the hydroxyl groups. Suitable acids used for esterification include fatty carboxylic acids having from about 4 to 30 carbon atoms, more preferably, fatty carboxylic acids having 6-22 carbon atoms. Examples of suitable sorbitan derivatives that may be used to form vesicles include PEG derivatives of sorbitan wherein the number of repeating ethylene oxide units ranges from 2 to 200, such as PEG sorbitan beeswax, PEG sorbitan lanolate, PEG sorbitan laurate, PEG sorbitan oleate, PEG sorbitan palmitate, PEG sorbitan perisostearate, PEG sorbitan peroleate, PEG sorbitan stearate, PEG sorbitan tetraoleate, glyceryl/sorbitol/oleate/hydroxystearate, PEG sorbitan cocoate, PEG sorbitan diisostearate, PEG sorbitan isostearate, PEG sorbitan tetrastearate, PEG sorbitan triisostearate; Also suitable are polysorbates, which are polymers from sorbitan. For example, Polysorbates 20 to 85 or Polysorbate 20 to 85 acetate are suitable, with the numbers 20 to 85 meaning the number of repeating sorbitan moieties. Sorbitan esters such as such as sorbitan caprylate, cocoate, diisostearate, dioleate, distearate, isostearate, laurate, oleate, olivate, palmitate, sesquiisostearate, sesquioleate, sesquistearate, stearate, triisostearate, trioleate and the like, may also be used to form vesicles.

Also suitable for the formation of vesicles are various types of glyceryl ethers, which are linear or branched ethers of polyglycerol which have the general formula:

R-(Gly)ₙ-OH

wherein n is 1-10 and R is a straight or branched, saturated or unsaturated alkyl having from about 6 to 30 carbon atoms, and Gly refers to the glycerol residue. Examples of suitable polyglyceryl derivatives include polyglyceryl isostearates, polyglyceryl caprates, polyglyceryl oleates, polyglyceryl dilinoleates, polyglyceryl dioleates, polyglyceryl diisostearates, polyglyceryl distearates, polyglyceryl isopalmitates, polyglyceryl laurates, and the like.

In one preferred embodiment of the invention, the association structures are small unilamellar vesicles, large unilamellar vesicles or multilamellar vesicles formed by PEG-12 glycerol dimyristate in aqueous media. The material for forming such vesicles is sold by Corwood Laboratories, Hauppage, N.Y., under the trademark QuSomes™, which are non-phospholipid polar lipids that form vesicles when contacted with aqueous media.

B. Liquid Crystals

Liquid crystals are formed when the composition comprises certain types of amphiphilic molecules that have polar and nonpolar portions. Such molecules orient in head-to-heat/tail-to-tail configuration to form either smectic or nematic liquid crystals that may be lyotropic. Liquid crystals differ from vesicles in that the lipid film does not self-close to form a blister or sac, but rather the liquid crystals exist in the appropriate molecular orientation in the liquid. The same polar lipid ingredients may be used to form liquid crystals as well as vesicles, and the formation of one versus the other depends on the polar lipids selected, the amount present, the solvent used (e.g. water or a volatile organic solvent) and various other parameters well known to one skilled in the art.

C. Micelles

The association structures may be present in the form of micelles which are formed when amphipathic molecules in aqueous media such as oil in water emulsion, organize so that the polar head groups of the amphiphilic molecule orient toward the continuous aqueous phase and the nonpolar tail groups of the amphiphilic molecules orient toward the dispersed oil phase. Micelles may be found in oil in water emulsions.

D. Reverse Micelles

The association structures may be in the form of reverse micelles. Reverse micelles are found in water in oil emulsions and occur when the polar head groups of the amphiphilic material orient toward the dispersed water droplets and the lipophilic portions toward the continuous lipophilic phase.

### III. Whitening Actives

Any whitening active may be incorporated into the association structures. Suggested ranges of whitening active are from about 0.001 to 95%, preferably from about 0.005 to 90%, more preferably from about 0.010 to 85% by weight of the total composition. Suitable whitening agents may act by inhibiting the enzyme tyrosinase, thereby inhibiting melanin production, or by exerting inhibitory effects on other pathways involved in production of skin melanin. Examples of suitable whitening agents include, but are not limited to the following.

A. Diphenylmethanes

Diphenylmethanes, including those set forth in U.S. Patent Application 2007/0098655 are suitable for use in the compositions and methods of the invention. Such diphenylmethanes are generally of the formula:

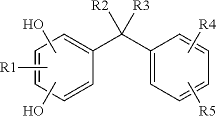

wherein:

R₁ is hydrogen, methyl, straight or branched saturated or unsaturated alkyl having 2 to 4 carbon atoms, OH, or halogen;

R₂ is hydrogen; methyl; straight or branched saturated or unsaturated alkyl having 2 to 5 carbon atoms;

R₃ is methyl, straight or branched saturated or unsaturated alkyl having 2 to 5 carbon atoms;

R₄ and R₅ are each independently hydrogen, methyl, straight or branched saturated or unsaturated alkyl having 2 to 5 carbon atoms;

and further wherein each of the substituents may assume any arbitrary position on the aromatic rings.

More preferred is where R₁ is hydrogen; R₂ is hydrogen or methyl; R3 is methyl; and R4 and R5 are each independently hydrogen or methyl.

Most preferred is wherein R₁ is hydrogen; R₂ is hydrogen; R₃ is methyl; and R₄ and R₅ are hydrogen and the compound is phenylethyl resorcinol.

B. Macrocyclic Compounds

Also suitable for use as the whitening active are macrocylic compounds as disclosed in U.S. Pat. No. 6,759,557, which is hereby incorporated by reference in its entirety. Such macrocyclic compounds have the general formula:

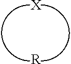

wherein X is selected from —CO—, —CHOH— and —CO—CHOH—; and wherein R is a hydrocarbon chain having from 1 to 24 carbon atoms and forming a ring with X. Preferably either of X and R is saturated or contains from 1 to 3 unsaturated bonds, and may be substituted with a lower alkyl group having from 1 to 10 carbon atoms. More preferred is where X is a carboxyl group.

Examples of such compounds include cyclotetradecanone, cyclopentadecanone, cyclohexadecanone, cycloheptadecanone, cyclooctadecanone, cyclononadecanone, cycloeicosanone, cycloheneicosanone, cyclodocosanone, cyclotricosanone, cyclotetracosanone, cyclopentacosanone, 3-methylcyclopentadecanone, (S)-3-methylcyclopentadecanone,®-3-methylcyclopentadecanone, 3-methylcyclohexadecanone, 4-methylcyclohexadecanone, 4-cyclopentadecenone, 5-cyclopentadecenone, 4-cyclohexadecenone, 5-cyclohexadecenone, (E)-5-cyclohexadecenone, (Z)-5-cyclohexadecenone, 9-cyclopentadecenone, (E)-9-cyclopentadecenone, (Z)-9-cyclopentadecenone, 3-methyl-4-cyclopentadecenone, 3-methyl-5-cyclopentadecenone, 3-methyl-4-cyclohexadecenone, 3-methyl-5-cyclohexadecenone, 4-methyl-4-cyclohexadecenone, 4-methyl-5-cyclohexadecenone, 10-cycloeicosenone, 11-cyclodocosenone and 12-cyclotetracosenone; cyclotetradecanol, cyclopentadecanol, cyclohexadecanol, cycloheptadecanol, cyclooctadecanol, cyclononadecanol, cycloeicosanol, cycloheneicosanol, cyclodocosanol, cyclotricosanol, cyclotetracosanol, cyclopentacosanol, 3-methylcyclopentadecanol, (1R,3R)-3-methylcyclopentadecanol, (1R,3S)-3-methylcyclopentadecanol, (1s,3R)-3-methylcyclopentadecanol, (1s,3S)-3-methylcyclopentadecanol, 3-methylcyclohexadecanol, (4-methylcyclohexadecanol, 4-cyclopentadecenol, 5-cyclopentadecenol, 4-cyclohexadecenol, 5-cyclohexadecenol, (E)-5-cyclohexadecenol, (S)-5-cyclohexadecenol, 9-cycloheptadecenol, (E)-9-cycloheptadecenol, (S)-9-cycloheptadecenol, 3-methyl-4-cyclopentadecenol, 3-methyl-5-cyclohexadecenol, 4-methyl-4-cyclohexadecenol, 4-methyl-5-cyclohexadecenol, 10-cycloeicosenol, 11-cyclodocosenol and 12-cyclotetracosenol; 2-hydroxycyclohexadecanone, 2-hydroxycycloheptadecanone, 2-hydroxycyclooctadecanone, 2-hydroxycyclononadecanone, 2-hydroxycycloeicosanone, 2-hydroxycycloheneicosanone, 2-hydroxycyclodocosanone, 2-hydroxycyclotricosanone, 2-hydroxycyclotetracosanone, 2-hydroxycycloheptacosanone, 2-hydroxycyclohexacosanone, 2-hydroxycyclo-3-methylcycloeicosanone, 2-hydroxy-20-methylcycloeicosanone, 2-hydroxy-4,19-dimethylcycloeicosanone, (4R)-2-hydroxy-4-methylcycloeicosanone, (19R)-2-hydroxy-19-methylcycloeicosanone, 2-hydroxy-8-cyclohexadecenone, 2-hydroxy-9-cycloheptadecenone, 2-hydroxy-10-cyclooctadecenone, 2-hydroxy-10-cyclononadecenone, 2-hydroxy-11-cycloeicosenone, (Z)-2-hydroxy-11-cycloeicosenone, (E)-2-hydroxy-11-cycloeicosenone, 2-hydroxy-10-cycloheneicosenone, 2-hydroxy-11-cyclodocosenone, 2-hydroxy-13-cyclotetracosenone, 2-hydroxy-3-methyl-11-cycloeicosenone, 2-hydroxy-20-methyl-11-cycloeicosenone, 2-hydroxy-4,19-dimethyl-11-cycloeicosenone, (4S)-2-hydroxy-4-methyl-11-cycloeicosenone, (19S)-2-hydroxy-19-methyl-11-cycloeicosenone, (5E,15E)-2-hydroxy-5,15-cyclooctadecadienone, (5E,17E)-2-hydroxy-4,19-dimethyl-5,17-cycloeicosadienone; 2-hydroxycycloeicosanone, 2-hydroxycycloheneicosanone, 2-hydroxy-11-cycloeicosenone, 2-hydroxy-11-cycloheneicosenone and 2-hydroxy-12-cycloheneicosenone; 2-hydroxycycloeicosanone, 2-hydroxycycloheneicosanone, 2-hydroxy-11-cycloeicosenone, 2-hydroxy-11-cycloheneicosenone and 2-hydroxy-12-cycloheneicosenone.

Typically, the macrocyclic compounds may be prepared by first preparing a corresponding unsaturated chain hydrocarbon having 20 or 21 carbon atoms, whose both end carbons form esterified carboxy groups; subjecting said esters to an acyloin condensation, so that an unsaturated macrocyclic compound is obtained; and optionally, subjecting said unsaturated macrocyclic compound to subsequent hydrogenation. One particularly preferred macrocyclic whitening agent is cyclohexadecanol.

C. Other Whitening Ingredients

Other whitening ingredients include botanical extracts that contain components that inhibit melanin production in skin such as licorice extract; pomegranate extract; hinokitiol; protocatechuic acid; NAB asafetida (Ferula Foetida) extract; resveratrol and his derivatives such as oxyresveratrol, resveratrol, resveratrol phosphate, resveratrol ferulate; ferulic acid and its derivatives such as ferulic acid phosphate; viniferol; botanical extract combinations sold by Coletica under the Phytoclar® (Saxifrage, Grape, mulberry and Scutelleria Root extracts), Phytowhite® (cucumber, apple and Scutellaria extracts) or Phytolight® (cucumber, apple and Scutellaria, and green tea extracts); Lunawhite B® (butylene glycol/water/Denothera Biennis seed extract) evening primrose extract; fatty acid esters of ascorbic acid such as ascorbyl palmitate; Euphrasia Officianalis extract, purine derivatives such as kinetin or derivatives thereof; ascorbyl glucoside; grape seed extract; vineferol, pomegranate extract, tetrahydrocurcumins, Acmella Oleracea extract, Aloesin, Tyrostat®, which are extracts of field dock, aspergillus ferment, molasses, and combinations of these ingredients.

In short, any whitening ingredient would be suitable for incorporation in to the association structures provided it is stable and compatible with the ingredients used to prepare the association structures.

### IV. Other Ingredients

In addition to the whitening active and association structures, the compositions used in the method of the invention may contain a variety of other ingredients.

The compositions of the invention may be in an aqueous solution or suspension form, or in the water-in-oil or oil-in-water emulsion form. In the case where the composition is aqueous based, the amount of water may range from about 0.1-99%, preferably from about 5-85%, more preferably from about 7-75% by weight of the total composition. In the case where the compositions are emulsions, the amount of oil will preferably range from about 1-95%, preferably from about 5-85%, more preferably from about 7-65% by weight of the total composition.

A. Aqueous Phase Structuring Agent

In the case where the compositions are in the form of aqueous solutions, dispersions or emulsions, in addition to water the aqueous phase may contain one or more aqueous phase structuring agents, that is, an agent that increases the viscosity or, or thickens, the aqueous phase of the composition. This is particularly desirable when the composition is in the form of a serum or gel. Suitable ranges of aqueous phase structuring agent, if present, are from about 0.01 to 30%, preferably from about 0.1 to 20%, more preferably from about 0.5 to 15% by weight of the total composition. Examples of such agents include various acrylate based thickening agents, natural or synthetic gums, polysaccharides, and the like, including but not limited to those set forth below. When the whitening active ingredient is in the water soluble form, the aqueous phase thickening agent also contributes to stabilizing this ingredient in the composition and improving penetration into the stratum corneum.

1. Polysaccharides

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

2. Acrylate Polymers

Also suitable are different types of synthetic polymeric thickeners. One type includes acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0.1-99 parts of the A monomer, and about 0.1-99 parts of the B monomer. Acrylic polymer solutions include those sold by Seppic, Inc., under the tradename Capigel.

Also suitable are acrylic polymeric thickeners that are copolymer of A, B, and C monomers wherein A and B are as defined above, and C has the general formula:

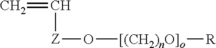

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

3. High Molecular Weight PEG or Polyglycerins

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.

B. Oils

In the event the compositions of the invention are in emulsion form, the composition will comprise an oil phase. Oily ingredients are desirable for the skin moisturizing and protective properties. Oils, if present, will form a barrier on the skin so that the whitening active ingredient present in the composition remains on the skin. Suitable oils include silicones, esters, vegetable oils, synthetic oils, including but not limited to those set forth herein. The oils may be volatile or nonvolatile, and are preferably in the form of a pourable liquid at room temperature. The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C.

1. Volatile Oils

Suitable volatile oils generally have a viscosity ranging from about 0.5 to 5 centistokes 25° C. and include linear silicones, cyclic silicones, paraffinic hydrocarbons, or mixtures thereof. Volatile oils may be used to promote more rapid drying of the skin care composition after it is applied to skin. Volatile oils are more desirable when the skin care products containing the whitening active ingredient are being formulated for consumers that have combination or oily skin. The term “combination” with respect to skin type means skin that is oily in some places on the face (such as the T-zone) and normal in others.

(a). Volatile Silicones

Cyclic silicones are one type of volatile silicone that may be used in the composition. Such silicones have the general formula:

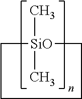

where n=3-6, preferably 4, 5, or 6.

Also suitable are linear volatile silicones, for example, those having the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

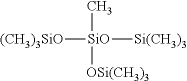

Methyl trimethicone may be purchased from Shin-Etsu Silicones under the tradename TMF-1.5, having a viscosity of 1.5 centistokes at 25° C.

(b). Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

2. Non-Volatile Oils

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to:

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.

(ii). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

(iii). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A. Cosmetic Ingredient Dictionary and Handbook, Eleventh Edition, 2006, under the classification of “Esters”, the text of which is hereby incorporated by reference in its entirety.

(b). Hydrocarbon Oils

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

(c). Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(d). Nonvolatile Silicones

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

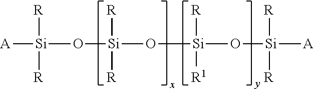

wherein R and R¹ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R¹ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the trade names Abil Wax 9801, or 9814.

(e). Fluorinated Oils

Various types of fluorinated oils may also be suitable for use in the compositions including but not limited to fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers include those disclosed in U.S. Pat. Nos. 5,183,589, 4,803,067, 5,183,588 all of which are hereby incorporated by reference, which are commercially available from Montefluos under the trademark Fomblin.

C. Oil Phase Structuring Agents

In the case where the composition is in the form of an emulsion, it may be desirable to include one or more oil phase structuring agents in the cosmetic composition. The term “oil phase structuring agent” means an ingredient or combination of ingredients, soluble or dispersible in the oil phase, which will increase the viscosity, or structure, the oil. The structuring agent may be present in an amount sufficient to provide a liquid composition with increased viscosity, a semi-solid, or in some cases a solid composition that may be self-supporting. The structuring agent itself may be present in the liquid, semi-solid, or solid form. Suggested ranges of structuring agent are from about 0.01 to 70%, preferably from about 0.05 to 50%, more preferably from about 0.1-35% by weight of the total composition. Suitable oil phase structuring agents include those that are silicone based or organic based. They may be polymers or non-polymers, synthetic, natural, or a combination of both.

1. Silicone Structuring Agents

A variety of oil phase structuring agents may be silicone based, such as silicone elastomers, silicone gums, silicone waxes, linear silicones having a degree of polymerization that provides the silicone with a degree of viscosity such that when incorporated into the cosmetic composition it is capable of increasing the viscosity of the oil phase. Examples of silicone structuring agents include, but are not limited to:

(a). Silicone Elastomers

Silicone elastomers suitable for use in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety. It is particularly desirable to incorporate silicone elastomers into the compositions of the invention because they provide excellent “feel” to the composition, are very stable in cosmetic formulations, and relatively inexpensive.

(b). Silicone Gums

Also suitable for use as an oil phase structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million.

The silicone gums that are used in the compositions include, but are not limited to, those of the general formula wherein:

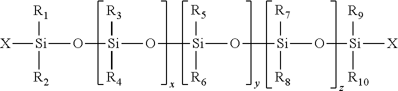

R₁ to R₉ are each independently an alkyl having 1 to 30 carbon atoms, aryl, or aralkyl; and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.

(c). Silicone Waxes

Another type of oily phase structuring agent includes silicone waxes that are typically referred to as alkyl silicone waxes which are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.

(d). Polyamides or Silicone Polyamides

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

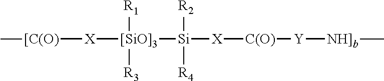

X is a linear or branched alkylene having from about 1-30 carbon atoms; R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

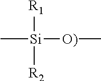

### and Y is:

(a) a linear or branched alkylene having from about 1-40 carbon atoms which may be substituted with:
(i) one or more amide groups having the general formula R₁CONR₁, or
(ii) C₅₋₆ cyclic ring, or
(iii) phenylene which may be substituted with one or more C₁₋₁₀ alkyl groups, or
(iv) hydroxy, or
(v) C₃₋₈ cycloalkane, or
(vi) C₁₋₂₀ alkyl which may be substituted with one or more hydroxy groups, or
(vii) C₁₋₁₀ alkyl amines; or

### (b) TR₅R₆R₇

wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

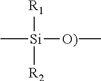

Preferred is where R₁, R₂, R₃, and R₄ are C₁₋₁₀, preferably methyl; and X and Y is a linear or branched alkylene. Preferred are silicone polyamides having the general formula

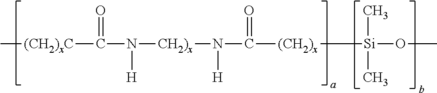

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.
Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

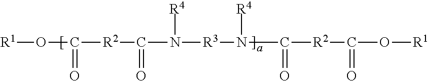

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C30-42 hydrocarbon; each R₃ is independently an organic group containing at least 2 carbon atoms, hydrogen atoms and optionally one or more oxygen or nitrogen atoms; and each R₄ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

(e). Natural or Synthetic Organic Waxes

Also suitable as the oil phase structuring agent may be one or more natural or synthetic waxes such as animal, vegetable, or mineral waxes. Preferably such waxes will have a higher melting point such as from about 50 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

(f). Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

(g). Silicas and Silicates

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

D. Surfactants

The composition may contain one or more surfactants, especially if in the emulsion form. Such surfactants may be silicone or organic based. The surfactants will aid in the formation of stable emulsions of either the water-in-oil or oil-in-water form. If present, the surfactant may range from about 0.001 to 30%, preferably from about 0.005 to 25%, more preferably from about 0.1 to 20% by weight of the total composition.

(1). Silicone Surfactants

Suitable silicone surfactants include polyorganosiloxane polymers that have amphiphilic properties, for example contain hydrophilic radicals and lipophilic radicals. These silicone surfactants may be liquids or solids at room temperature.

(a). Dimethicone Copolyols or Alkyl Dimethicone Copolyols

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

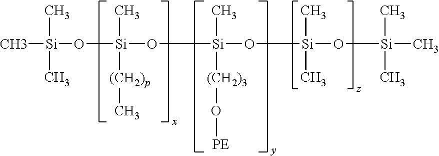

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

(b). Crosslinked Silicone Surfactants

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

(c). Organic Nonionic Surfactants

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Also suitable as nonionic surfactants are formed by the reaction of a carboxylic acid with an alkylene oxide or with a polymeric ether. The resulting products have the general formula: where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

E. Humectants

It may also be desirable to include one or more humectants in the composition. If present, such humectants may range from about 0.001 to 25%, preferably from about 0.005 to 20%, more preferably from about 0.1 to 15% by weight of the total composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₄ alkylene glycols, most particularly butylene glycol.

F. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including yeast ferment extract, Padina Pavonica extract, thermus thermophilic ferment extract, camelina sativa seed oil, boswellia serrata extract, olive extract, Aribodopsis Thaliana extract, Acacia Dealbata extract, Acer Saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, Panax Ginseng, Siegesbeckia Orientalis, Fructus Mume, Ascophyllum Nodosum, Bifida Ferment lysate, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, and mixtures thereof.

G. Sunscreens

It may also be desirable to include one or more sunscreens in the compositions of the invention. Such sunscreens include chemical UVA or UVB sunscreens or physical sunscreens in the particulate form. Inclusion of sunscreens in the compositions containing the whitening active ingredient will provide additional protection to skin during daylight hours and promote the effectiveness of the whitening active ingredient on the skin.

1. UVA Chemical Sunscreens

If desired, the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds having the general formula

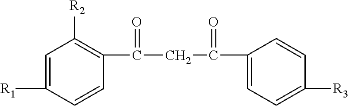

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

Other types of UVA sunscreens include dicamphor sulfonic acid derivatives, such as ecamsule, a sunscreen sold under the trade name Mexoryl™, which is terephthalylidene dicamphor sulfonic acid, having the formula:

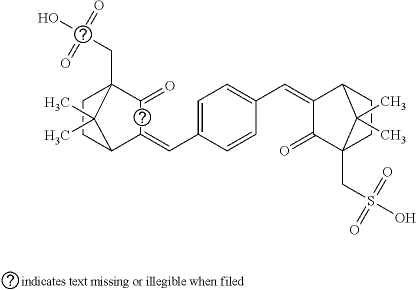

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

2. UVB Chemical Sunscreens

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

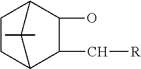

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

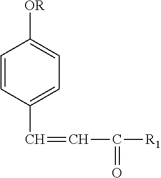

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula:

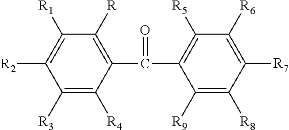

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

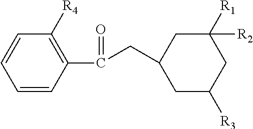

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

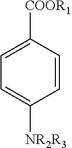

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

If desired, the compositions of the invention may be formulated to have a certain SPF (sun protective factor) values ranging from about 1-50, preferably about 2-45, most preferably about 5-30. Calculation of SPF values is well known in the art.

H. Particulate Materials

The compositions of the invention may contain particulate materials in the form of pigments, inert particulates, or mixtures thereof. If present, suggested ranges are from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

1. Powders

The particulate matter may be colored or non-colored (for example white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

2. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

I. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

J. Vitamins and Antioxidants

The compositions of the invention may contain vitamins and/or coenzymes, as well as antioxidants. If so, 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and so on; the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are retinyl palmitate, retinol. retinoic acid, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### V. The Cosmetic Compositions

The compositions of the invention containing the whitening active in association structures may be found in a variety of forms, such as aqueous based solutions, serums, gels, skin creams or lotions, or color cosmetic compositions such as foundation makeup, mascara, lip color, blush, eyeshadow, and the like.

If the composition is in the emulsion form, the whitening active in association structures may be found in the water phase or the oil phase of the emulsion depending on the type of association structure that has been formed. For example, certain lipids that are used are more hydrophilic than lipophilic and will generally exhibit a preference for the water phase of the emulsion. Certain other lipids are more lipophilic in nature and will exhibit a greater affinity for the oil phase of the emulsion.

Suitable serums or gels will generally comprise from about 1-99% water, and optionally from about 0.001-30% of an aqueous phase thickening agent. The other ingredients mentioned herein may be present in the percentage ranges set forth.

Typical skin creams or lotions comprise from about 5-98% water, 1-85% oil, and from about 0.1 to 20% of one or more surfactants. Preferably the surfactants are nonionic and may be in the form of silicones or organic nonionic surfactants.

Typical color cosmetic compositions such as foundations, blush, eyeshadow and the like will preferably contain from about 5-98% water, 1-85% oil, and from about 0.1 to 20% of one or more surfactants in addition to from about 0.1 to 65% of particulates that are pigments or a combination of pigments and powders.

Typical mascara compositions generally contain from about 5-98% water, 1-85% oil, and from about 0.1 to 20% surfactant in addition to natural or synthetic polymers that are film forming, such as aqueous dispersions of acrylic copolymers, aqueous dispersions of polyurethane, or silicone resins.

### VI. The Methods

The invention further comprises whitening or brightening skin by treating the skin with compositions of the invention. The compositions may be applied in the forms mentioned herein, as part of skin care regimens. For example, the composition may be applied to the skin as a night cream or cream applied to skin prior to a period of bodily rest such as a nap or sleep. The composition may be applied two times a day, in the morning and in the evening after cleansing the skin. The composition may be applied to the skin over skin care products, in the form of foundations or other color cosmetics.

In one embodiment, the whitening active in association structures is formulated into a day cream and a night cream, so that the consumer using the regimen applies the composition to the skin twice a day as part of a standard skin care routine.

In another embodiment, the whitening active in association structures is applied to the skin in the form of a toner, over which a skin cream or lotion is applied.

In another embodiment the whitening agent in association structures is applied to the skin in the form of a skin cleanser.

The invention will be further described in connection with the following examples which are set forth for the purposes of illustration only.

### Example 1

Skin treatment compositions were prepared as follows:

| | w/w % | | |
|---|---|---|---|
| Ingredients | 1 | 2 | 3 |
| Cholesterol/potassium sulfate | 0.20 | 0.20 | 0.20 |
| Selaginella Tamariscina (Spike Moss) | 0.002 | 0.002 | 0.002 |
| extract | | | |
| Wheat (Triticum Vulgare) bran | 0.20 | 0.20 | 0.20 |
| extract/olive (Olea Europaea) extract | | | |
| Vinyl dimethicone/methicone | 13.00 | 13.00 | 13.00 |
| silsesquioxane crosspolymer | | | |
| Hydrogenated lecithin | 1.00 | 1.00 | 1.00 |
| Nordihydroguaiaretic acid | 0.001 | 0.001 | 0.001 |
| Cholesterol | 0.20 | 0.20 | 0.20 |
| Acetyl glucosamine | 2.00 | 2.00 | 2.00 |
| Phenylethyl resorcinol | | 1.00 | 0.50 |
| Adipic acid/neopentyl glycol | | 5.00 | |
| crosspolymer/water/dimethicone/ | | | |
| hydryoxypropyl methylcellulose/VP/VA | | | |
| crosspolymer/amondimethicone | | | |
| Ascorbyl glucoside | 2.00 | 2.00 | 2.00 |
| Dimethicone | 2.00 | 2.00 | 2.00 |
| Ammonium acrylodimethyltaurate/VP | 1.10 | 1.10 | 1.10 |
| copolymer | | | |
| Humulus Lupulus (Hops) extract/Linoleic | 0.005 | 0.005 | 0.005 |
| acid/Linolenic acid | | | |
| Rosemary extract | 0.002 | 0.002 | 0.002 |
| Phenoxyethanol | 0.005 | 0.005 | 0.005 |
| Disodium EDTA | 0.10 | 0.10 | 0.10 |
| Ethylhexylglycerin | 0.30 | 0.30 | 0.30 |
| Dipotassium glycyrrhizate | 0.20 | 0.20 | 0.20 |
| Pentylene glycol | 1.00 | 1.00 | 1.00 |
| Dimethicone crosspolymer-3/isododecane | 2.00 | 2.00 | 2.00 |
| Water/Acetyl hexapeptide-8 | 1.00 | 1.00 | 1.00 |
| Butylene glycol | 3.00 | 3.00 | 3.00 |
| Punica Granatum juice extract | 0.001 | 0.001 | 0.001 |
| Arginine | 0.95 | 0.95 | 0.95 |
| Cyclodextrin/ethylbisiminomethylguaiacol | 0.001 | 0.001 | 0.001 |
| manganese chloride | | | |
| Vitis Vinifera (Grape) seed extract | 0.002 | 0.002 | 0.002 |
| Caprylyl glyol/phenoxyethanol/hexylene | 0.50 | 0.50 | 0.50 |
| glycol | | | |
| Polyglyceryl-3 disiloxane dimethicone | 0.50 | 0.50 | 0.50 |
| Silica | 7.00 | 7.00 | 7.00 |
| Citri Reticulatae peel extract | 0.001 | 0.001 | 0.001 |
| Simethicone | 0.0001 | 0.0001 | 0.0001 |
| Malt extract | 0.14 | 0.14 | 0.14 |
| Polygonum Cuspidatum root extract | 0.005 | 0.005 | 0.005 |
| Trametes Versicolor extract | 2.00 | 2.00 | 2.00 |
| Water | QS | QS | QS |
| | w/w % | | |
| Ingredients | 4 | 5 | 6 |
| Cholesterol/potassium sulfate | 0.20 | 0.20 | 0.20 |
| Selaginella Tamariscina (Spike Moss) | 0.002 | 0.002 | 0.002 |
| extract | | | |
| Wheat (Triticum Vulgare) bran | 0.20 | 0.20 | 0.20 |
| extract/olive (Olea Europaea) extract | | | |
| Vinyl dimethicone/methicone | 13.00 | 13.00 | 13.00 |
| silsesquioxane crosspolymer | | | |
| Hydrogenated lecithin | 1.00 | 1.00 | 1.00 |
| Nordihydroguaiaretic acid | 0.001 | 0.001 | 0.001 |
| Lauryl PEG-9 polydimethylsiloxyethyl | | 1.50 | 1.50 |
| dimethicone | | | |
| Cholesterol | 0.20 | 0.20 | 0.20 |
| Acetyl glucosamine | 2.00 | 2.00 | 2.00 |
| Phenylethyl resorcinol | 0.10 | | |
| 9 parts PEG-12 glyceryl dimyristate, | | 5.00 | 5.00 |
| 1 part phenylethyl resorcinol | | | |
| Adipic acid/neopentyl glycol | | | 2.00 |
| crosspolymer/water/dimethicone/ | | | |
| hydryoxypropyl methylcellulose/VP/VA | | | |
| crosspolymer/amondimethicone | | | |
| Ascorbyl glucoside | 2.00 | 2.00 | 2.00 |
| Dimethicone | 2.00 | 2.00 | 2.00 |
| Ammonium acrylodimethyltaurate/VP | 1.10 | 1.10 | 1.10 |
| copolymer | | | |
| Humulus Lupulus (Hops) extract/Linoleic | 0.005 | 0.005 | 0.005 |
| acid/Linolenic acid | | | |
| Rosemary extract | 0.002 | 0.002 | 0.002 |
| Phenoxyethanol | 0.005 | 0.015 | 0.015 |
| Disodium EDTA | 0.10 | 0.10 | 0.10 |
| Ethylhexylglycerin | 0.30 | 0.30 | 0.30 |
| Dipotassium glycyrrhizate | 0.20 | 0.20 | 0.20 |
| Pentylene glycol | 1.00 | 1.00 | 1.00 |
| Dimethicone crosspolymer-3/isododecane | 2.00 | 2.00 | 2.00 |
| Water/Acetyl hexapeptide-8 | 1.00 | 1.00 | 1.00 |
| Butylene glycol | 3.00 | 3.00 | 3.00 |
| Punica Granatum juice extract | 0.001 | 0.001 | 0.001 |
| Arginine | 0.95 | 0.95 | 0.95 |
| Cyclodextrin/ethylbisiminomethylguaiacol | 0.001 | 0.001 | 0.001 |
| manganese chloride | | | |
| Vitis Vinifera (Grape) seed extract | 0.002 | 0.002 | 0.002 |
| Caprylyl glyol/phenoxyethanol/hexylene | 0.50 | 0.50 | 0.50 |
| glycol | | | |
| Polyglyceryl-3 disiloxane dimethicone | 0.50 | 0.50 | 0.50 |
| Silica | 7.00 | 7.00 | 7.00 |
| Citri Reticulatae peel extract | 0.001 | 0.001 | 0.001 |
| Simethicone | 0.0001 | 0.0001 | 0.0001 |
| Malt extract | 0.14 | 0.04 | 0.04 |
| Polygonum Cuspidatum root extract | 0.005 | 0.005 | 0.005 |
| Trametes Versicolor extract | 2.00 | 0.0002 | 0.0002 |
| Black strap molasses | | 0.20 | 0.20 |
| Water | QS | QS | QS |

Compositions 1-4 were prepared by combining the water and oil phase ingredients separately and emulsifying. Composition 5 was prepared by pre-mixing 9 parts of PEG-12 glycerol dimyristate and 1 part phenyl ethyl resorcinol to form multilamellar vesicles. The remaining oil phase and water phase ingredients were separately combined and mixed well to form an emulsion. The pre-mix was added. Composition 6 was prepared by combining 9 parts of PEG-12 glyceryl dimyristate and 1 part phenylethyl resorcinol to form multilamellar lipid vesicles in a pre-mix. The remaining oil and water phase ingredients were separately combined and mixed well to form an emulsion. The pre-mix was added.

### Example 2

Compositions 1-6 were tested on skin by conducting a sting test. Subjects suitable for participation in the study were selected. Using a sterile cotton tipped applicator a solution of 10% lactic acid in distilled water was applied to the suborbital, malar, and naso-labial fold area on one side of the face (5 even strokes) while U.S.P. physiological saline was applied to the other side of the face. Subjects were asked to identify the perceived degree of stinging on each side of the face 2.5 and 5.0 minutes after application of the materials according to the following table:

| Score | Sting Perception |
|---|---|
| 0 | None |
| 1 | Slight sting |
| 2 | Moderate sting |
| 3 | Intense, severe sting |

Subjects were instructed to wash their face and were released. Subjects who reported a score of “3” or higher were selected for participation in the study. Ten subjects were selected and placed in an environmental chamber having a temperature of about 100° F. and 80% relative humidity. After profuse facial sweating occurred, a solution of 10% lactic acid was applied to facial skin in the suborbital, malar, and naso-labial fold areas of the face using a cotton tipped applicator and applying five even strokes. Attribution of sting potential was based on the following scale where n equals the combined stinging scores of all ten subjects at both 2.5 and 5.0 minutes:

<10 Very slight potential for stinging

10-19 Slight potential for stinging

20-25 Slight to moderate potential for stinging

>25 Strong potential for stinging

The results were as follows. The sting test result is graded on a 0.1 to 10 basis with 0.1 being the best and 10 being the worse (most stinging):

| Composition No. | Sting Test Result |
|---|---|
| 1 (negative control) | 0-0/10 panelists reported |
| | stinging out of 10 tested |
| 2 (1% whitening active) | 5.1-10/10 |
| 3 (0.5% whitening active) | 2.8-9/10 |
| 4 (0.1% whitening active) | 0.5-3/10 |
| 5 (5% of a mixture of 1 part | 0-0/10 |
| whitening active in 9 parts of | |
| PEG-12 glyceryl dimyristate | |
| vesicle = 0.5% whitening | |
| active applied to skin) | |
| 6 (5% of a mixture of 2 part | 0.4-1/10 |
| whitening active in 8 parts of | |
| PEG-12 glyceryl dimyristate | |
| vesicle = 1% whitening | |
| active applied to skin) | |

The test scores are interpreted as follows. For example, 0-0/10 means that the sum of the 2.5 and 5.0 minute scores for all the panelists was 0, and that 0 panelists reporting a perception of stinging and that a total of 10 panelists were tested. For the score 5.1-10/10, the sum of the 2.5 minute and 5.0 minute sting test scores were added for all ten panelists tested and that number divided by ten. The second digit after the dash refers to the number of panelists who reported a perception of stinging, in this case 10. The third digit after the “/” refers to the number of panelists tests. Similarly for the score 2.8-9/10, the 2.8 refers to the sum of 2.5 and 5.0 minute sting test scores for all ten panelists divided 10. The “9” refers to the number of panelists who reported the perception of stinging, and the digit “10” refers to the total number of panelists tested.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A method for reducing skin irritation of a skin whitening active by formulating the whitening active in liquid crystals formed from a PEG glyceryl fatty (C4-30) acid ester where the whitening active is selected from the group consisting of those having the formula: 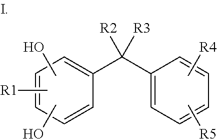
wherein:
R₁ is hydrogen, methyl, straight or branched saturated or unsaturated alkyl having 2 to 4 carbon atoms, OH, or halogen;
R₂ is hydrogen; methyl; straight or branched saturated or unsaturated alkyl having 2 to 5 carbon atoms;
R₃ is methyl, straight or branched saturated or unsaturated alkyl having 2 to 5 carbon atoms;
R₄ and R₅ are each independently hydrogen, methyl, straight or branched saturated or unsaturated alkyl having 2 to 5 carbon atoms;
and further wherein each of the substituents may assume any arbitrary position on the aromatic rings; 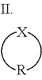
wherein X is selected from —CO—, —CHOH— and —CO—CHOH—; and wherein R is a hydrocarbon chain having from 1 to 24 carbon atoms and forming a ring with X. Preferably either of X and
R is saturated or contains from 1 to 3 unsaturated bonds, and may be substituted with a lower alkyl group having from 1 to 10 carbon atoms.

2. The method of claim 1 wherein the skin irritation that is reduced is skin stinging.

3. The method of claim 2 wherein the reduction in skin stinging is measured by the skin sting test score.

4. The method of claim 1 wherein the PEG glyceryl fatty (C4-30) acid ester is PEG glyceryl mono- or dimyristate.

5. The method of claim 4 wherein the PEG glyceryl mono- or dimyristate is PEG-12 glyceryl dimyristate.

6. The method of claim 2 wherein the liquid crystal consists essentially of the PEG glyceryl fatty (C4-30) acid ester and the whitening active.

7. The method of claim 2 wherein the liquid crystal consists of the PEG glyceryl fatty (C4-30) acid ester and the whitening active.

8. A method for making a reduced irritation topical skin whitening composition with a skin whitening agent selected from octyldodecanol or phenethyl resorcinol by or mixtures thereof by formulating the whitening active in liquid crystals formed from a PEG glyceryl fatty (C4-30) acid ester.
